(19) United States
(12) Patent Application Publication
(10) Pub. No.: US 2006/0073102 A1
(43) Pub. Date: Apr. 6, 2006

(54) Abuse-resistant opioid solid dosage form

(76) Inventors: Kao D Huaihung, Svosset, NY (US); Bradley Galer, West Chester, PA (US); David Kerr, Sparta, NJ (US)

Correspondence Address: DILWORTH & BARRESE, LLP, 333 EARLE OVINGTON BLVD., UNIONDALE, NY, 11553, US

(21) Appl. No.: 10/514,387

(22) PCT Filed: May 13, 2003
(86) PCT No.: PCT/US03/14840

§ 371 Date: Aug. 15, 2005

Related U.S. Application Data
(60) Provisional application No. 60/453,700, filed on May 13, 2002.

(51) Int. Cl. A61K 9/44

(52) U.S. Cl. 424/10.2; 424/422

(57) ABSTRACT

The present invention pertains to a solid dosage form comprising an analgesically effective amount of opioid analgesic and an opioid abuse-deterring amount of a nontoxic N-methyl-D-aspartate receptor antagonist contained in a carrier which isolates, or separates, the antagonist from the opioid analgesic. The nontoxic N-methyl-D-aspartate receptor antagonist is released and made available only when the dosage form is misused, as would be the case when the dosage form is crushed or dissolved and thereafter administered in a manner other than that indicated, e.g., by injection or intranasally.

### CROSS-REFERENCE TO RELATED APPLICATIONS

This application claims the benefit under 35 U.S.C. § 119(e) of earlier filed and copending U.S. Provisional Application No. 60/453,700, filed May 13, 2002, the contents of which are incorporated by reference herein.

### BACKGROUND OF THE INVENTION

1. Field of the Invention

The present invention relates to abuse-resistant opioid compositions. More particularly, the present invention relates to abuse-resistant opioid-containing solid dosage pharmaceuticals comprising an analgesically effective amount of an opioid analgesic in combination with an opioid euphoria-inhibiting amount of an isolated nontoxic N-methyl-D-aspartate receptor antagonist which is substantially not released when the dosage form is administered intact.

2. Description of the Related Art

Morphine, a classic opioid, has been known as a very powerful analgesic compound for many years. Its potential as a target of abuse has been known for almost as long. Opioids and their derivatives are used in the pharmaceutical industry as narcotic analgesics, hypnotics, sedatives, anti-diarrheals, anti-spasmotics, and antitussives. Despite their well known potential for addiction and abuse, opioids are widely used due to their superior, powerful analgesic properties.

In the past, abuse of opioids was generally limited to illicit drugs made in illegal laboratories. Abuse of pharmaceutical opioids was quite limited. Accordingly, action by makers of pharmaceutical opioids would, in the past, have little or no effect on illegal abuse of opioids.

Recently, however, this trend has been changing and abuse of pharmaceutical opioids has been increasing. This is especially true in the case of extended release opioid dosage forms. One reason for the increase of abuse is that extended release opioid dosage forms are intended for decreased frequency of dosing, which results in the production of dosage forms having substantially increased amounts of opioid. Therefore, an extended release dosage form, such as a tablet for oral administration, can provide much more opioid to the potential abuser than the past low dose, immediate release dosage forms.

There have previously been attempts in the art to control the abuse potential associated with opioid analgesics. Typically, a particular dose of an opioid analgesic is more potent when administered parenterally as compared to the same dose administered orally. Therefore, one popular mode of abuse of oral medications involves the extraction of the opioid from the dosage form, and the subsequent injection of the opioid (using any “suitable” vehicle for injection) in order to achieve a “high.” Attempts to curtail abuse have therefore typically centered around the inclusion in the oral dosage form of an opioid antagonist which is not orally active but which will substantially block the analgesic effects of opioid if one attempts to dissolve the opioid and administer it parenterally.

Other attempts to control the abuse of opioids have combined opioids and/or opioid agonists with opioid antagonists in a dosage form which separates the two and only releases the opioid antagonist if abused. For example, U.S. Pat. No. 5,149,538, the contents of which are incorporated by reference herein, discloses an abuse-resistant dosage form for the transdermal delivery of opioids whereby the opioid is combined with an opioid antagonist that is separated from the opioid by an impermeable barrier that will release the opioid antagonist upon ingestion or immersion of the transdermal device in a solvent.

Similarly, WO 01/58451, the contents of which are also incorporated by reference herein, discloses an oral dosage form containing an opioid agonist in releasable form and a sequestered opioid antagonist which is not released when the dosage form is administered intact, but is released if the oral dosage form is tampered with.

GB 1 390 772, the contents of which are incorporated by reference herein, discloses a narcotic composition for oral administration which includes a narcotic which has substantial activity both orally and by injection, in combination with a narcotic antagonist which is much less effective orally than by injection. Therefore, the antagonist has little effect when the tablet is taken orally as intended. However, the opioid antagonists have substantially increased effect when taken directly into the blood stream. Thus, abusing the opioid by dissolving or crushing the tablet, and then ingesting same by injecting or snorting (intranasal administration), would cause the antagonist to have its full effect, essentially blocking the opioid receptors, preventing the abuser from receiving an opioid effect, and inducing withdrawal in opioid-dependent individuals.

N-methyl-D-aspartate (NMDA) receptor antagonists are well known in the art and encompass, for example, dextromethorphan, dextrorphan, memantine, amantidine, d-methadone and their pharmaceutically acceptable salts. NMDA receptor antagonists are known to inhibit the development of tolerance to and/or dependence on addictive drugs, e.g., narcotic analgesics such as morphine, codeine, etc., as described in U.S. Pat. Nos. 5,321,012 and 5,556,838, and to treat chronic pain as described in U.S. Pat. No. 5,502,058, the contents of each of which are incorporated by reference herein.

Controlled release dosage forms, for pharmaceuticals, which include extended release and sustained release dosage forms, are known to those skilled in the art. See, e.g., U.S. Pat. Nos. 4,861,598, 4,970,075, 5,266,331, 5,508,042, 5,549,912, 5,656,295, 5,958,459, 5,968,551, 6,103,261, 6,143,322, 6,143,353, and 6,294,195, the contents of each of which are incorporated by reference herein. For example, U.S. Pat. Nos. 4,861,598 and 4,970,075 disclose controlled release pharmaceutical compositions for oral administration having extended action due to their use of a higher aliphatic alcohol and acrylic resin as their base material. Pharmaceutically active agents utilized with these compositions include narcotics. U.S. Pat. Nos. 5,266,331, 5,508,042, 5,549,912 and 5,656,295 disclose solid controlled release oral dosage forms of oxycodone or its salts whereby the oxycodone is encompassed in a carrier with a defined dissolution rate for the extended release of the pharmaceutical in vitro.

With the increase in the abuse of extended release opioid compositions, it would be beneficial to develop a dosage form which would make abuse more difficult and less desirable for opioid abusers.

### BRIEF SUMMARY OF THE INVENTION

The present invention relates to an abuse-resistant opioid-containing solid dosage form comprising an analgesically effective amount of an opioid analgesic and an isolated nontoxic N-methyl-D-aspartate antagonist which is substantially not released when the dosage form is administered intact, said nontoxic N-methyl-D-aspartate receptor antagonist being present in an opioid euphoria-inhibiting amount. The nontoxic N-methyl-D-aspartate antagonist can be released very slowly or not at all when the solid dosage form is taken as intended, but altering the dosage form will result in the full release of the nontoxic N-methyl-D-aspartate antagonist which, because of its dysphoric effects, will prevent or discourage abuse. In addition, if abused intranasally, the nontoxic N-methyl-D-aspartate antagonist will act as an irritant to the nasal passages and thus prevent or discourage nasal abuse of the dosage form.

With oral and nasal abuse, abusers chew or crush a controlled release opioid tablet to convert the tablet to immediate release. Abusers then take the crushed tablet orally or intranasally (by snorting the powder) in order to obtain a euphoria or high. Thus, the solid dosage form of the present invention will prevent nasal and oral abuse of orally administered controlled release solid dosage forms, which are becoming much more commonly abused.

If the solid dosage form is dissolved and injected, the NMDA receptor antagonist will prevent the abuser from receiving a euphoric high. This is due both to the increased efficacy of the antagonist when injected, as well as to the high doses of antagonist released by the crushed solid dosage form. Thus, the solid dosage form of the present invention should prevent abuse by administration of the dosage in any altered form, whether crushed or dissolved, and whether swallowed, snorted, or injected.

### DETAILED DESCRIPTION OF THE INVENTION

The solid dosage form in accordance with the present invention comprises an opioid analgesic in combination with an opioid euphoria-inhibiting amount of a nontoxic NMDA receptor antagonist. The NMDA receptor antagonist, in turn, is present in a substantially non-releasable form, that is, it is isolated within a carrier which provides a reduced release rate or little or no release of the NMDA receptor antagonist when the solid dosage form is administered as intended. Thus, the NMDA receptor antagonist has little or no effect on the desired analgesia from the opioid when the dosage form is taken as intended and does not pose a risk of precipitating withdrawal in opioid tolerant or dependent patients. However, should the solid dosage form be altered for the purposes of abuse, e.g., crushed or dissolved in water or some other aqueous solvent, the NMDA receptor antagonist will be released in an amount that will inhibit the euphoria produced by the opioid.

The solid dosage form of the present invention may be administered orally, transdermally, rectally or topically.

The terms “alter”, “altered”, or “altering” mean any manipulation by mechanical, thermal and/or chemical means which changes the physical properties of the dosage form, e.g. to liberate the opioid analgesic for immediate release if it is in sustained release form, or to make the opioid analgesic available for inappropriate use such as administration by an alternate route, e.g., parenterally. The dosage form can be altered, e.g., by means of crushing, shearing, grinding, chewing, dissolution in a solvent, heating (e.g., greater than about 45° C.), or any combination thereof.

For purposes of this disclosure, the expression “opioid euphoria-inhibiting” includes the suppression, cloaking, masking or countering of the euphoria-inducing properties of opioids, e.g., by a mechanism of dysphoria.

The term “carrier” includes any material, composition or device that physically separates and isolates the N-methyl-D-aspartate receptor antagonist from the opioid analgesic and impedes or prevents the release of the N-methyl-D-aspartate receptor antagonist when the dosage form is taken as intended, i.e., without alteration of its form, but releases the N-methyl-D-aspartate receptor antagonist in an opioid euphoria-inhibiting amount when the dosage form is altered.

For purposes of this disclosure, “controlled release” includes “extended release” and “sustained release” and pertains to the release of pharmaceutical agents at a defined level over an extended period of time.

The expression “dosage form” is understood to include “unit dosage form”. The expression “unit dosage form” means a physically discrete unit which contains specified amounts of the opioid analgesic and nontoxic NMDA receptor antagonist, in combination with a carrier and/or any other pharmacologically active substance or pharmaceutical excipient, which amounts are selected so that a fixed number, e.g. one, of the units is suitable to achieve a desired therapeutic effect.

The term “an isolated nontoxic opioid euphoria-inhibiting N-methyl-D-aspartate receptor antagonist which is substantially not released” refers to a nontoxic NMDA receptor antagonist that is not released or substantially not released after the intact dosage form containing both opioid analgesic and the nontoxic NMDA receptor antagonist is administered intact (e.g., without having been altered). Such a dosage form is also referred to as comprising an “isolated antagonist”.

Although the preferred embodiments of the invention comprise a nontoxic NMDA receptor antagonist in a form that completely prevents the release of the nontoxic NMDA receptor antagonist, the invention also includes an antagonist in a substantially non-releasable form. The term “substantially not released” refers to the antagonist that might be released in a small amount, as long as the amount released does not significantly adversely affect analgesic efficacy when the dosage form is administered to humans as intended.

The first component of the abuse-resistant opioid-containing pharmaceutical solid dosage form is an analgesically effective amount of an opioid analgesic. Opioid analgesics suitable for use in the solid dosage form generally have a potential for abuse and include, but are not limited to, alfentanil, allyiprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethyhnethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

The preferred dosage of opioid analgesic can range from about 1 mg per 70 kg body weight of subject to about 800 mg per 70 kg body weight per unit dose. Preferably, the dosage of opioid analgesic is from about 10 mg per 70 kg body weight to about 500 mg per 70 kg body weight in the unit dosage form. Where the opioid analgesic is fentanyl or sufentanyl, the preferred dosage is from about 5 μg per 70 kg to about 250 μg per 70 kg body weight per unit dose.

The second component of the abuse-resistant opioid-containing pharmaceutical solid dosage form is an opioid euphoria inhibiting amount of nontoxic opioid euphoria-inhibiting NMDA receptor antagonist in a slow-release or non-release frangible and/or water soluble carrier. Nontoxic opioid euphoria-inhibiting NMDA receptor antagonists suitable for use in accordance with the present invention include dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), d-methadone (d-form of 6-dimethylamino-4,4-diphenyl-3-heptanone hydrochloride), their mixtures and their pharmaceutically acceptable salts. Dextromethorphan is a preferred NMDA receptor antagonist due to its ready availability and wide acceptance as an ingredient of many over-the-counter medications where it is utilized for its cough-suppressant (antitussive) activity. Not only will the dextromethorphan inhibit or diminish the euphoria-producing effects of the opioid but, when the dosage form is abused intranasally, it will also act as an irritant to the nasal mucosa and thus prevent or deter or inhibit abuse of the opioid by intranasal administration.

The term “nontoxic” as used herein shall be understood in a relative sense and is intended to designate any substance that has been approved by the United States Food and Drug Administration (“FDA”) for administration to humans or, in keeping with established regulatory criteria and practice, is susceptible to approval by the FDA for administration to humans. The term “nontoxic” is also used herein to distinguish the NMDA receptor antagonists that are useful in the practice of the present invention from NMDA receptor antagonists such as MK 801 (the compound 5-methyl-10,11-dihydro-SH-dibenze[a,d] cyclohepten-5,10-imine), CPP (the compound 3-[2-carboxypiperazin-4-yl] propyl-1-phosphonic acid) and PCP (the compound 1-(1-phenylcyclohexyl) piperidine) whose toxicities effectively preclude their therapeutic use.

The amount of NMDA receptor antagonist can vary, but is in an opioid euphoria-inhibiting amount. In some instances, the NMDA receptor antagonist may be in an amount sufficient to induce withdrawal. The dosage of nontoxic NMDA receptor antagonist can range from about 100 mg per 70 kg body weight to about 500 mg per 70 kg body weight per unit dose. Preferably, the dosage of nontoxic NMDA receptor antagonist is from about 200 mg per 70 kg body weight to about 400 mg per 70 kg body weight, with a range of about 225 mg per 70 kg body weight to about 325 mg per 70 kg body weight being most preferred in the unit dosage form. While any NMDA receptor antagonist may be used, in a preferred embodiment dextromethorphan is used.

The nontoxic NMDA receptor antagonist must be present in the combined dosage form in an opioid euphoria-inhibiting amount. It would be recognized by one skilled in the art that this will relate to the particular opioid analgesic present and its euphoria-inducing capacity which, in turn, is believed to be related to its abuse potential. The amount of nontoxic NMDA receptor antagonist for combination with a specific opioid analgesic in a particular combined unit dosage form will depend upon the nature and amount of the opioid and its euphoria-inducing capacity and the nature of the nontoxic NMDA receptor antagonist and its ability to produce an opioid euphoria-inhibiting effect, as well as the particular formulation containing the active substances and the state and circumstances of the host being treated. As those skilled in the art will recognize, many factors that modify the action of the active substances herein will be taken into account by the treating physician such as the age, body weight, sex, diet and condition of the subject, the time of administration, the rate and route of administration, and so forth. Optimal dosages for a given set of conditions can be ascertained by those skilled in the art using conventional dosage determination tests. Table 1 below sets forth ranges for several specific opioid analgesics and a preferred nontoxic NMDA receptor antagonist, dextromethorphan.

In certain embodiments, an opioid antagonist is included in the carrier in addition to the nontoxic NMDA receptor antagonist and, like the NMDA receptor antagonist, is only released in the event the solid dosage form is altered. Suitable opioid antagonists include naltrexone, naloxone, nalmephene, cyclazocine, levallorphan, and mixtures thereof.

Additionally, the solid dosage form herein can optionally contain at least one other pharmacologically active substance e.g., an analgesically useful amount of a non-narcotic analgesic such as acetaminophen, nonsteroidal anti-inflammatory drug (NSAID) such as aspirin, bromfenac, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and the like, cyclooxygenase-II (COX II) inhibitor such as celecoxib (Celebrex), rofecoxib (Vioxx), meloxicam, L-745337 (Merck), MK-966 (Merck), L-768277 (Merck), GR-253035 (Glaxo-Wellcome), JTE-S22 (Japan Tobacco), RS-57067-000 (Roche), SC-58125 (Searle), SC-078 (Searle), PD-138387 (Warner-Lambert), NS-398 (Taisho), flosulide and PD-164387 (Warner-Lambert), or other COX-II inhibitor such as any of those described in, e.g., U.S. Pat. Nos. 5,616,601; 5,604,260; 5,593,994; 5,550,142; 5,536,752; 5,521,213; 5,474,995; 5,639,780; 5,604,253; 5,552,422; 5,510,368; 5,436,265; 5,409,944; and 5,130,311, all of which are hereby incorporated by reference.

The carrier containing and isolating the NMDA receptor antagonist impedes or prevents the release of the antagonist under normal circumstances (i.e., where the solid dosage form is administered as intended), but releases the antagonist where the solid dosage form is altered. The carrier containing the NMDA receptor antagonist can be formed in many ways. It is preferred to use a carrier comprising a base material made of hydrophilic polymers, hydrophobic polymers, long chain hydrocarbons, polyalkylene glycols, higher aliphatic alcohols, acrylic resins, and mixtures thereof.

In one embodiment, the pharmaceutical dosage form comprises a sustained release carrier. Alternatively, a normal release carrier having a coating that controls the release of the drug may be used. Suitable base materials for controlled release carriers include combinations of higher aliphatic alcohols and acrylic resins.

Base compositions prepared from such higher aliphatic alcohols and acrylic resins provide sustained release of therapeutically active ingredients over a period of time from five hours and for as much as 24 hours after administration, generally oral administration, in humans or animals.

These bases can be prepared from any pharmaceutically acceptable higher aliphatic alcohol, the most preferred being fatty alcohols of 10-18 carbon atoms, particularly stearyl alcohol, cetyl alcohol, cetostearyl alcohol, lauryl alcohol, myristyl alcohol and mixtures thereof.

Any acrylic polymer which is pharmaceutically acceptable can be used for the purposes of the present invention. The acrylic polymers may be cationic, anionic or non-ionic polymers and may be acrylates, methacrylates, formed of methacrylic acid or methacrylic acid esters. These polymers can be synthesized, as indicated above, to be cationic, anionic or non-ionic, which then renders the polymers that would be pH dependent and consequently soluble in, or resistant to solutions over a wide range in pH.

In addition, suitable materials for inclusion in a controlled release carrier include:

(a) Hydrophilic polymers, such as gums, cellulose ethers, acrylic resins and protein derived materials. Of these polymers, the cellulose ethers, especially hydroxyalkylcelluloses and carboxyalkylcelluloses, are preferred. The dosage form may contain between 1% and 80% (by weight) of at least one hydrophilic or hydrophobic polymer.

(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between 25° and 90° C. are preferred. Of these long chain hydrocarbon materials, fatty (aliphatic) alcohols are preferred. The oral dosage form may contain up to 60% (by weight) of at least one digestible, long chain hydrocarbon.

(c) Polyalkylene glycols. The oral dosage form may contain up to 60% (by weight) of at least one polyalkylene glycol.

One particularly suitable carrier comprises at least one water soluble hydroxyalkyl cellulose, at least one C₁₂-C₃₆, preferably C₁₄-C₂₂, aliphatic alcohol and, optionally, at least one polyalkylene glycol.

The at least one hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present pharmaceutical dosage form will be determined, inter alia, by the precise rate of opioid analgesic release required. Preferably however, the oral dosage form contains between 1% and 45%, especially between 5% and 25% (by weight) of the at least one hydroxyalkyl cellulose.

While the at least one aliphatic alcohol may be, for example, lauryl alcohol, myristyl alcohol or stearyl alcohol, in particularly preferred embodiments the at least one aliphatic alcohol is cetyl alcohol or cetostearyl alcohol. The amount of the at least one aliphatic alcohol in the present dosage form will be determined, as above, by the precise rate of opioid analgesic release required. It will also depend on whether at least one polyalkylene glycol is present in or absent from the dosage form. In the absence of at least one polyalkylene glycol, the dosage form preferably contains between 20% and 50% (by weight) of the at least one aliphatic alcohol. When at least one polyalkylene glycol is present in the dosage form, then the combined weight of the at least one aliphatic alcohol and the at least one polyalkylene glycol preferably constitutes between 20% and 50% (by weight) of the total dosage.

In the present preferred dosage form, the ratio of e.g., the at least one hydroxyalkyl cellulose or acrylic resin to the at least one aliphatic alcohol/polyalkylene glycol determines, to a considerable extent, the release rate of the opioid analgesic from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or polyethylene glycol, which is preferred. The number average molecular weight of the at least one polyalkylene glycol is preferred between 1000 and 15000 especially between 1500 and 12000.

Another suitable controlled release carrier would comprise an alkylcellulose (especially ethyl cellulose), a C₁₂ to C₃₆ aliphatic alcohol and, optionally, a polyalkylene glycol.

In addition to the above ingredients, a controlled release carrier may also contain suitable quantities of other materials, e.g. diluents, lubricants, binders, granulating aids, colorants, flavorants and glidants that are conventional in the pharmaceutical art.

As an alternative to a controlled release carrier, the present carrier may be a normal release carrier having a coat that controls the release of the drug. In particularly preferred embodiments of this aspect of the invention, the present dosage form comprises film coated spheroids containing active ingredient and a non-water soluble spheronising agent. The term spheroid is known in the pharmaceutical art and means a spherical granule having a diameter of between 0.5 mm and 2.5 mm especially between 0.5 mm and 2 mm.

The spheronising agent may be any pharmaceutically acceptable material that, together with the active ingredient, can be spheronised to form spheroids. Microcrystalline cellulose is preferred. According to a preferred aspect of the present invention, the film coated spheroids contain between 70% and 99% (by wt), especially between 80% and 95% (by wt), of the spheronising agent, especially microcrystalline cellulose.

In addition to the active ingredient and spheronising agent, the spheroids may also contain a binder. Suitable binders, such as low viscosity, water soluble polymers, will be well known to those skilled in the pharmaceutical art. However, water soluble hydroxy lower alkyl cellulose, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose.

The spheroids are preferably film coated with a material that permits release of the opioid analgesic at a controlled rate in an aqueous medium. The film coat is chosen so as to achieve, in combination with the other ingredients, the in-vitro release rate outlined above (between 12.5% and 42.5% (by weight) release after 1 hour, etc.).

The film coat will generally include a water insoluble material such as: (a) a wax, either alone or in admixture with a fatty alcohol; (b) shellac or zein; (c) a water insoluble cellulose, especially ethyl cellulose; (d) a polymethacrylate.

Preferably, the film coat comprises a mixture of the water insoluble material and a water soluble material. The ratio of water insoluble to water soluble material is determined by, amongst other factors, the release rate required and the solubility characteristics of the materials selected.

The water soluble material may be, for example, polyvinylpyrrolidone or, which is preferred, a water soluble cellulose, especially hydroxypropylmethyl cellulose.

Suitable combinations of water insoluble and water soluble materials for the film coat include shellac and polyvinylpyrrolidone or, which is preferred, ethyl cellulose and hydroxypropylmethyl cellulose.

In another embodiment, in order to obtain a sustained-release of the opioid sufficient to provide an analgesic effect for the extended durations set forth in the present invention, the substrate comprising the therapeutically active agent may be coated with a sufficient amount of hydrophobic material to obtain a weight gain level from about 2 to about 30 percent, although the overcoat may be greater depending upon the physical properties of the particular opioid analgesic compound utilized and the desired release rate, among other things.

The solvent which is used for the hydrophobic material may be any pharmaceutically acceptable solvent, including water, methanol, ethanol, methylene chloride and mixtures thereof. It is preferable however, that the coatings be based upon aqueous dispersions of the hydrophobic material.

In certain preferred embodiments of the present invention, the hydrophobic polymer comprising the sustained-release coating is a pharmaceutically acceptable acrylic polymer, including but not limited to acrylic acid and methacrylic acid copolymers, methacrylic acid copolymers, methyl methacrylate copolymers, ethoxyethyl methacrylates, cynaoethyl methacrylate, methyl methacrylate, copolymers, methacrylic acid copolymers, methyl methacrylate copolymers, methyl methacrylate copolymers, methyl methacrylate copolymers, methacrylic acid copolymer, aminoalkyl methacrylate copolymer, methacrylic acid copolymers, methyl methacrylate copolymers, poly(acrylic acid), poly(methacrylic acid, methacrylic acid alkylamide copolymer, poly(methyl methacrylate), poly(methacrylic acid) (anhydride), methyl methacrylate, polymethacrylate, methyl methacrylate copolymer, poly(methyl methacrylate), poly(methyl methacrylate) copolymer, polyacrylamide, aminoalkyl methacrylate copolymer, poly(methacrylic acid anhydride), and glycidyl methacrylate copolymers.

In other preferred embodiments, the hydrophobic polymer which may be used for coating the substrates of the present invention is a hydrophobic cellulosic material such as ethylcellulose. Those skilled in the art will appreciate that other cellulosic polymers, including other alkyl cellulosic polymers, may be substituted for part or all of the ethylcellulose included in the hydrophobic polymer coatings of the present invention.

In embodiments of the present invention where the coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is especially preferred.

Examples of suitable plasticizers for the acrylic polymers of the present invention include citric acid esters such as triethyl citrate NF XVI, tributyl citrate, dibutyl phthalate, and possibly 1,2-propylene glycol, polyethylene glycols, propylene glycol, diethyl phthalate, castor oil, and triacetin, although it is possible that other water-insoluble plasticizers (such as acetylated monoglycerides, phthalate esters, castor oil, etc.) may be used. Triethyl citrate is especially preferred.

The sustained-release profile of the formulations of the invention can be altered, for example, by varying the thickness of the hydrophobic coating, changing the particular hydrophobic material used, or altering the relative amounts of, e.g., different acrylic resin lacquers, altering the manner in which the plasticizer is added (e.g., when the sustained-release coating is derived from an aqueous dispersion of hydrophobic polymer), by varying the amount of plasticizer relative to hydrophobic polymer, by the inclusion of additional ingredients or excipients, by altering the method of manufacture, etc.

Sustained-release spheroids or beads, coated with a therapeutically active agent are prepared, e.g. by dissolving the opioid analgesic in water and then spraying the solution onto a substrate using a Wurster insert. Optionally, additional ingredients are also added prior to coating the beads in order to assist the opioid analgesic binding to the substrates, and/or to color the solution, etc. For example, a product which includes hydroxypropyl methylcellulose, etc. with or without colorant may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic sustained-release coating. An example of a suitable barrier agent is one which comprises hydroxypropyl methylcellulose. However, any film-former known in the art may be used. It is preferred that the barrier agent does not affect the dissolution rate of the final product.

The coating solutions of the present invention may contain, in addition to the film-former, plasticizer, and solvent system (i.e., water), a colorant to provide elegance and product distinction. Color may be added to the solution of the therapeutically active agent instead, or in addition to the aqueous dispersion of hydrophobic polymer.

The plasticized aqueous dispersion of hydrophobic polymer may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic polymer to obtain a predetermined sustained-release of said therapeutically active agent when said coated substrate is exposed to aqueous solutions, e.g. gastric fluid, is preferably applied, taking into account the physically characteristics of the therapeutically active agent, the manner of incorporation of the plasticizer, etc. After coating with the hydrophobic polymer, a further overcoat of a film-former is optionally applied to the beads. This overcoat is provided, if at all, in order to substantially reduce agglomeration of the beads.

Next, the coated beads are cured in order to obtain a stabilized release rate of the therapeutically active agent.

One appropriate configuration for the solid dosage form is a uniform controlled release carrier with the NMDA receptor antagonist dispersed therein. The controlled release carrier is formulated with the NMDA receptor antagonist and granulated into very small granules. These granules are then incorporated into the main carrier of the solid dosage form. In this way, the NMDA receptor antagonist is contained in a separate controlled release carrier which forms part of the solid dosage form. Upon ingestion, the principle carrier of the solid dosage form, which contains the opioid analgesic, dissolves, releasing the opioid analgesic and also releasing the granules containing the NMDA receptor antagonist in a controlled release or non-release carrier. The granules then pass through and out of the body, releasing only minimal NMDA receptor antagonist, or no NMDA receptor antagonist at all.

Another configuration for the solid dosage form of the present invention is one in which the NMDA receptor antagonist is incorporated into an immediate release carrier. The carrier is then granulated and coated with a non-release coating, such as an acrylic polymer.

The granules are then incorporated into a controlled release solid dosage form. Upon administration, the solid dosage form releases the opioid at a predetermined rate, but the coated granules do not release the NMDA receptor antagonist. Rather, the granules pass through the intestines and are eliminated from the patient. In this way, the coated granules act as an excipient and will, under normal circumstances, have no pharmacological effect whatsoever. Any suitable controlled release carrier can be used for the NMDA receptor antagonist, provided that the proper non-release coating is used along with it.

Alternatively, granules having a reduced release rate could be formed using an immediate release carrier with a reduced release rate coating over the granules. This is acceptable as long as the release rate is very low (lower than necessary to antagonize the therapeutic effect of the opioid analgesic when the dosage form is taken as intended). Thus, “non-release” as used herein includes any reduced release carrier which allows less than about 30 percent of the NMDA receptor antagonist to be released over about a 12-hour period under normal conditions of oral administration.

Furthermore, a suitable non-release coating may be formed by using several known coatings together on a granulated carrier-containing NMDA receptor antagonist. For instance, the antagonist granules can be covered with a coating which allows for release of material only at a pH below about 5, which is then covered by a coating which allows release of material only at a pH above about 5. It is preferred to coat the antagonist granules with a coating that allows release of material at a pH below about 3, which is then covered with a coating that allows release of material at a pH above about 7, or even more preferably, above about 9. In this way, when the solid dosage form is ingested, the outer coating will prevent release of material while the granules reside in the stomach, and the inner coating will prevent release of material once the solid dosage form has passed through the stomach into the intestines, where the pH rises sufficiently to dissolve the outer coating.

The NMDA receptor antagonist need not be fully encapsulated so as to be inert. It may be desirable to allow some release of the NMDA receptor antagonist to provide relief from the side effects of the opioid analgesic if small amounts of the NMDA receptor antagonist will enhance the opioid analgesic's effectiveness. Thus, the encapsulation can provide variable release of the NMDA receptor antagonist depending on the formulation.

Moreover, the slow-release or non-release carrier containing the NMDA receptor antagonist may be a barrier which is slowly permeable or impermeable to the NMDA receptor antagonist. Such barrier may be made of or contain a material such as polyethylene, polypropylene, ethylene/propylene copolymer, ethylene/ethylacrylate copolymer, ethylene/vinyl acetate copolymer, silicone elastomer, medical-grade polydimethylsiloxane, neoprene rubber, polyisobutylene, chlorinated polyethylene, polyvinyl chloride, vinyl chloride-vinyl acetate copolymer, polymethacrylate polymer, polyvinylidene chloride, polyethylene terephathalate, butyl rubber, epichlorohydrin rubber, ethylene-vinyl alcohol copolymer, ethylenevinyloxyethanol copolymer, silicone copolymer, cellulose polymer, polycarbonate, polytetrafluoroethylene, starch, gelatin, natural or synthetic gum and their mixtures.

Generally, the amount of NMDA receptor antagonist used in the solid dosage form of the present invention will vary with the amount and type of opioid analgesic used. Listed below in Table 1 are some examples of the combined opioid analgesic and NMDA receptor antagonist that can be utilized in accordance with the present invention. It should be understood that any numerical value provided is approximate and should be construed that to mean approximately or about that number.

TABLE
SOLID DOSAGE FORMS

| | OPIOID ANALGESIC, | NMDA RECEPTOR |
|---|---|---|
| | mg per 70 kg body | ANTAGONIST, mg per 70 kg |
| EXAMPLE | weight per unit dose | body weight per unit dose |
| 1 | codeine, 5-360 | dextromethorphan HBr, 5-500 |
| 2 | dihydrocodeine, 2-200 | dextromethorphan HBr, 5-500 |
| 3 | hydrocodone, 2-400 | dextromethorphan HBr, 5-500 |
| 4 | hydromorphone, 4-64 | dextromethorphan HBr, 10-500 |
| 5 | morphine, 5-800 | dextromethorphan HBr, 10-500 |
| 6 | oxycodone, 5-400 | dextromethorphan HBr, 10-500 |
| 7 | oxymorphone, 2-100 | dextromethorphan HBr, 10-500 |
| 8 | tramadol, 25-200 | dextromethorphan HBr, 10-250 |
| 9 | propiram, 25-200 | dextromethorphan HBr, 5-500 |

It will be understood that various modifications may be made to the embodiments disclosed herein. Therefore, the above description should not be construed as limiting, but merely as exemplifications of preferred embodiments. For example, NMDA receptor antagonist other than dextromethorphan can be utilized in the solid dosage form described herein. Those skilled in the art will envision other modifications within the scope and spirit of the claims appended hereto.

What is claimed is:

1. An abuse-resistant opioid-containing pharmaceutical solid dosage form which comprises:
a) an analgesically effective amount of opioid analgesic; and,
b) an isolated nontoxic N-methyl-D-aspartate receptor antagonist which is substantially not released when the dosage form is administered intact, but is released in an opioid euphoria-inhibiting amount when the dosage form is crushed or dissolved and then administered.

2. The dosage form of claim 1 wherein the opioid analgesic is at least one member selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

3. The dosage form of claim 1 wherein the opioid analgesic is at least one member selected from the group consisting of codeine, dihydrocodeine, hydrocodone, hydromorphone, levorphanol, meperidine, methadone, morphine, oxycodone, oxymorphone, propoxyphene and their pharmaceutically acceptable salts.

4. The dosage form of claim 1 wherein the nontoxic NMDA receptor antagonist is at least one member selected from the group consisting of dextromethorphan, dextrorphan, memantine, amantidine, d-methadone and their pharmaceutically acceptable salts.

5. The dosage form of claim 3 wherein the nontoxic NMDA receptor antagonist is at least one member selected from the group consisting of dextromethorphan, dextrorphan, memantine, amantidine, d-methadone and their pharmaceutically acceptable salts.

6. The dosage form of claim 1 wherein the opioid analgesic is in a controlled release carrier.

7. The dosage form of claim 6 wherein the controlled release carrier comprises a base material selected from the group consisting of hydrophilic polymers, hydrophobic polymers, long chain hydrocarbons, polyalkylene glycols, higher aliphatic alcohols, acrylic resins, and mixtures thereof.

8. The dosage form of claim 1 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

9. The dosage form of claim 1 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

10. The dosage form of claim 1 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

11. The dosage form of claim 6 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

12. The dosage form of claim 6 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

13. The dosage form of claim 6 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

14. The dosage form of claim 7 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

15. The dosage form of claim 7 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

16. The dosage form of claim 7 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

17. The dosage form of claim 1 wherein the slow-release or non-release carrier is a barrier which is slowly permeable or impermeable to the nontoxic N-methyl-D-aspartate receptor antagonist.

18. The dosage form of claim 17 wherein the barrier is, or contains, a material selected from the group consisting of polyethylene, polypropylene, ethylene/propylene copolymer, ethylene/ethylacrylate copolymer, ethylene/vinyl acetate copolymer, silicone elastomer, medical-grade polydimethylsiloxane, neoprene rubber, polyisobutylene, chlorinated polyethylene, polyvinyl chloride, vinyl chloride-vinyl acetate copolymer, polymethacrylate polymer, polyvinylidene chloride, polyethylene terephathalate, butyl rubber, epichlorohydrin rubber, ethylene-vinyl alcohol copolymer, ethylenevinyloxyethanol copolymer, silicone copolymer, cellulose polymer, polycarbonate, polytetrafluoroethylene, starch, gelatin, natural or synthetic gum and their mixtures.

19. The dosage form of claim 1 further comprising an isolated opioid antagonist which is substantially not released when the dosage form is administered intact.

20. The dosage form of claim 19 wherein the opioid antagonist is selected from the group consisting of naltrexone, naloxone, nalmephene, cyclazocine, levallorphan, and mixtures thereof.

21. An abuse-resistant opioid-containing pharmaceutical solid dosage form which comprises:
a) an analgesically effective amount of at least one opioid analgesic selected from the group consisting of codeine, dihydrocodeine, hydrocodone, hydromorphone, levorphanol, meperidine, methadone, morphine, oxycodone, oxymophone, propoxyphene and their pharmaceutically acceptable salts; and,
b) an isolated amount of dextromethorphan which is substantially not released when the dosage form is administered intact, said dextromethorphan being present in an opioid euphoria-inhibiting amount.

22. The dosage form of claim 21 wherein the opioid analgesic is in a controlled release carrier.

23. The dosage form of claim 22 wherein the controlled release carrier is selected from the group consisting of hydrophilic polymers, hydrophobic polymers, long chain hydrocarbons, polyalkylene glycols, higher aliphatic alcohols, acrylic resins, and mixtures thereof.

24. The dosage form of claim 21 wherein the slow-release or non-release carrier is a barrier which is slowly permeable or impermeable to the dextromethorphan.

25. The dosage form of claim 22 wherein the slow-release or non-release carrier is a barrier which is slowly permeable or impermeable to the dextromethorphan.

26. The dosage form of claim 23 wherein the slow-release or non-release carrier is a barrier which is slowly permeable or impermeable to the dextromethorphan.

27. The dosage form of claim 21 wherein the opioid analgesic is present in an amount of from about 1 mg to about 800 mg per 70 kg body weight per unit dose and the dextromethorphan is present in an amount of from about 100 mg to about 500 mg per 70 kg body weight per unit dose.

28. The dosage form of claim 21 wherein the opioid analgesic is present in an amount of from about 10 mg to about 500 mg per 70 kg body weight per unit dose and the dextromethorphan is present in an amount of from about 200 mg to about 400 mg per 70 kg body weight per unit dose.

29. The dosage form of claim 21 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

30. The dosage form of claim 22 wherein the opioid analgesic is present in an amount of from about 1 mg to about 800 mg per 70 kg body weight per unit dose and the dextromethorphan is present in an amount of from about 100 mg to about 500 mg per 70 kg body weight per unit dose.

31. The dosage form of claim 22 wherein the opioid analgesic is present in an amount of from about 10 mg to about 500 mg per 70 kg body weight per unit dose and the dextromethorphan is present in an amount of from about 200 mg to about 400 mg per 70 kg body weight per unit dose.

32. The dosage form of claim 22 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

33. The dosage form of claim 23 wherein the opioid analgesic is present in an amount of from about 1 mg to about 800 mg per 70 kg body weight per unit dose and the dextromethorphan is present in an amount of from about 100 mg to about 500 mg per 70 kg body weight per unit dose.

34. The dosage form of claim 23 wherein the opioid analgesic is present in an amount of from about 10 mg to about 500 mg per 70 kg body weight per unit dose and the dextromethorphan is present in an amount of from about 200 mg to about 400 mg per 70 kg body weight per unit dose.

35. The dosage form of claim 23 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.

36. The dosage form of claim 21 further comprising an isolated opioid antagonist which is substantially not released when the dosage form is administered intact.

37. The dosage form of claim 36 wherein the opioid antagonist is selected from the group consisting of naltrexone, naloxone, nalmephene, cyclazocine, levallorphan, and mixtures thereof.

38. A solid opioid-containing pharmaceutical solid dosage form which is resistant to abuse by intranasal administration which comprises:
a) an analgesically effective amount of opioid analgesic; and,
b) an isolated nontoxic N-methyl-D-aspartate receptor antagonist which is substantially not released when the dosage form is administered intact but is released in a nasal mucosa-irritating amount when the dosage form is crushed or dissolved and then administered intranasally.

39. The dosage form of claim 38 wherein the opioid analgesic is at least one member selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, sufentanyl, tilidine, tramadol and their pharmaceutically acceptable salts.

40. The dosage form of claim 38 herein the opioid analgesic is at least one member selected from the group consisting of codeine, dihydrocodeine, hydrocodone, hydromorphone, levorphanol, meperidine, methadone, morphine, oxycodone, oxymorphone, propoxyphene and their pharmaceutically acceptable salts.

41. The dosage form of claim 38 herein the nontoxic NMDA receptor antagonist is at least one member selected from the group consisting of dextromethorphan, dextrorphan, memantine, amantidine, d-methadone and their pharmaceutically acceptable salts.

42. The dosage form of claim 40 wherein the nontoxic NMDA receptor antagonist is at least one member selected from the group consisting of dextromethorphan, dextrorphan, memantine, amantidine, d-methadone and their pharmaceutically acceptable salts.

43. The dosage form of claim 38 wherein the opioid analgesic is present in an amount of from about 1 mg to about 800 mg per 70 kg body weight per unit dose and the nontoxic NMDA receptor antagonist is present in an amount of from about 100 mg to about 500 mg per 70 kg body weight per unit dose.

44. The dosage form of claim 38 wherein the opioid analgesic is present in an amount of from about 10 mg to about 500 mg per 70 kg body weight per unit dose and the nontoxic NMDA receptor antagonist is present in an amount of from about 200 mg to about 400 mg per 70 kg body weight per unit dose.

45. The dosage form of claim 38 wherein the opioid analgesic is selected from the group consisting of fentanyl and sufentanyl and is present in an amount of from about 5 μg to about 250 μg per 70 kg body weight per unit dose and the nontoxic NMDA receptor antagonist is present in an amount of from about 100 mg to about 500 mg per 70 kg body weight per unit dose.
